(19) United States
(12) Patent Application Publication
(10) Pub. No.: US 2026/0088131 A1
(43) Pub. Date: Mar. 26, 2026

(54) SYSTEM AND METHOD FOR EVALUATING MOLECULAR PROPERTIES

(71) Applicant: Tesorai, Inc., San Diego, CA (US)

(72) Inventors: Maximilien Burq, San Diego, CA (US); Peter Cimermancic, San Diego, CA (US)

(73) Assignee: Tesorai, Inc., San Diego, CA (US)

(21) Appl. No.: 19/340,668

(22) Filed: Sep. 25, 2025

Related U.S. Application Data
(60) Provisional application No. 63/699,240, filed on Sep. 26, 2024.

(51) Int. Cl. G16B 40/00; G16B 50/00

(52) U.S. Cl. CPC G16B 40/00; G16B 50/00

(57) ABSTRACT

The method can include: determining a set of molecules, determining a representation for the set of molecules, and evaluating a property of the set of molecules. In variants, the method can function to evaluate molecules (e.g., pairs of molecules). For example, the method can function to identify high-potential hits (e.g., biologically relevant interactions between molecules) and/or other targets. Additionally or alternatively, the method can function to identify candidate molecules with a target property.

### CROSS-REFERENCE TO RELATED APPLICATIONS

This application claims the benefit of US Provisional Application number 63/699,240 filed 26, Sep. 2024, which is incorporated in its entirety by this reference.

### TECHNICAL FIELD

This invention relates generally to the molecular analysis field, and more specifically to a new and useful system and method for evaluating molecular properties in the molecular analysis field.

### BRIEF DESCRIPTION OF THE SEVERAL VIEWS OF THE DRAWINGS

FIG. 1 is a schematic representation of a variant of the method.

FIGS. 2A-2C are schematic representations of examples of the method.

FIGS. 3A-3B are schematic representations of examples of the method, including evaluating candidate molecules.

FIGS. 4A-4B are schematic representations of examples of encoder training.

FIG. 5 is a schematic representation of an example of model training.

FIG. 6 is a schematic representation of a specific example of the method, including processing experimental data.

FIG. 7A is an illustrative example of an image for a screening plate, including one pixel for each well of the screening plate.

FIG. 7B is an illustrative example of a transformed image for a screening plate (e.g., a transformation of the image in FIG. 7A).

FIG. 7C is another illustrative example of an image for a screening plate.

FIG. 7D is another illustrative example of a transformed image for a screening plate (e.g., a transformation of the image in FIG. 7C).

FIG. 7E is another illustrative example of an image for a screening plate.

FIG. 7F is another illustrative example of a transformed image for a screening plate (e.g., a transformation of the image in FIG. 7E).

### DETAILED DESCRIPTION OF THE INVENTION

The following description of the embodiments of the invention is not intended to limit the invention to these embodiments, but rather to enable any person skilled in the art to make and use this invention.

### 1. Overview

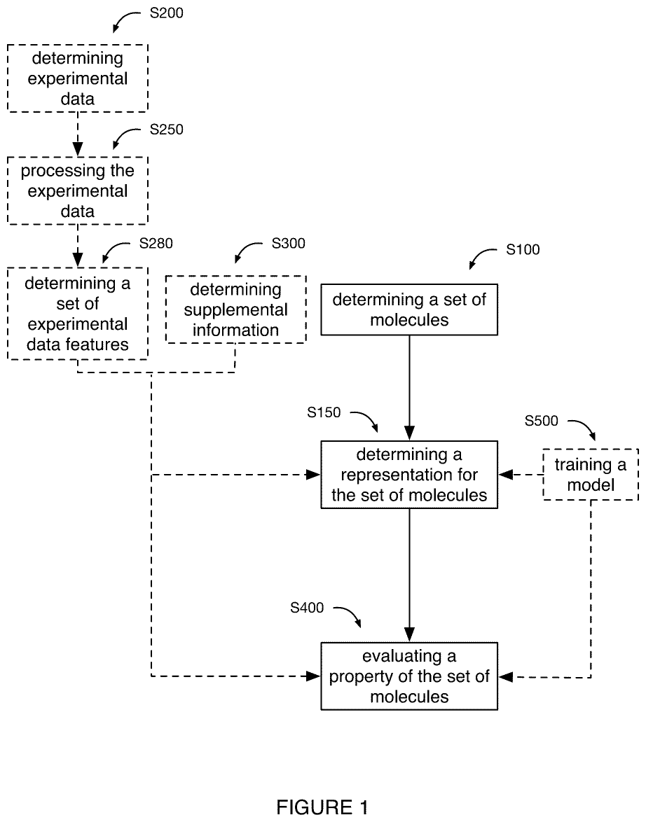

As shown in FIG. 1, the method can include: determining a set of molecules S100, determining a representation for the set of molecules S150, and evaluating a property of the set of molecules S400. However, the method can additionally or alternatively include any other suitable steps.

In variants, the method can function to prioritize experimental measurements of molecule interactions and/or other molecule properties. For example, the method can function to identify high-potential hits (e.g., biologically relevant interactions between molecules) and/or other targets. Additionally or alternatively, the method can function to identify candidate molecules with a target property. However, the method can otherwise function to evaluate molecule properties.

### 2. EXAMPLES

In a first example, the method can include: receiving experimental data for a property of a set of molecules (e.g., an experimentally observed hit); processing the experimental data to remove experimental noise (e.g., batch and plate effects); determining an embedding for the set of molecules using one or more molecule encoders (e.g., based on molecule sequence, structure, etc.); and, using an evaluation model, outputting a score for the property (e.g., a likelihood that the hit is biologically relevant) based on: the embedding, the experimental data (e.g., a set of experimental data features calculated based on the processed experimental data), and optionally supplementary information. The set of molecules can be a single molecule of interest, a pair of molecules (e.g., a pair of interacting molecules), and/or include any number of molecules. In a specific example, the experimental data can include screening plate images, where each image contains one or more pixels corresponding to each well on the screening plate (e.g., where each well corresponds to a pair of molecules).

In a first specific example, for a set of molecules that includes a pair of molecules, determining the embedding for the set of molecules can include determining an individual embedding for each molecule in the pair, and determining an aggregate embedding based on the individual embeddings (e.g., concatenated individual embeddings, a pairwise embedding output from a molecule encoder based on the individual embeddings, etc.). In a second specific example, for a set of molecules that includes a pair of molecules, determining the embedding for the set of molecules can include directly determining a pairwise embedding for the pair of molecules. In illustrative examples, the property of the set of molecules can include: a quantified interaction between the set of molecules (e.g., an abundance of a pair of molecules interacting), a quantified presence and/or abundance of the set of molecules, thermostability, cell permeability, a biomarker, and/or any other property of one or more molecules. The method can optionally include ranking multiple properties and/or molecules based on the respective scores. The method can optionally include identifying a subset of multiple properties and/or multiple molecules (e.g., multiple sets of molecules) based on the respective scores. In an illustrative example, identified sets of molecules can include pairs of molecules that are predicted to have a meaningful (e.g., biologically relevant) interaction.

In a second example, the method can include: determining candidate molecules; for each candidate molecule, determining an embedding (using one or more molecule encoders) for a pair of molecules that includes a molecule of interest and the candidate molecule; using an evaluation model, outputting a score for each pair of molecules (e.g., a likelihood that pair of molecules will bind or otherwise interact) based on the embedding and optionally supplementary information; identifying a subset of the candidate molecules (e.g., associated with the highest scoring pairs of molecules) and/or ranking the candidate molecules based on the respective scores. In an illustrative example, the identified candidate molecules can include small molecules that are predicted to bind to the molecule of interest.

However, the system / method can be otherwise performed.

### 3. Technical Advantages

Variants of the technology can confer one or more advantages over conventional technologies.

First, current experimental analysis systems identify potential hits (e.g., from high throughput screening assays) with a high rate of false positives and negatives; inefficient and expensive validation assays are then needed to identify the true hits. Variants of the technology can increase efficiency and decrease costs by increasing the probability of identifying high-potential hits and/or other targets in experimental data. In examples, high-potential hit identification can be used for: drug development (e.g., drug target identification); identification of allergens, new enzymes, and/or other molecules; biomarker identification; and/or other uses. In an example, meaningful (e.g., biologically relevant) interactions between molecules can be distinguished from noise (e.g., observed interactions that are not biologically relevant) with higher accuracy. In specific examples, variants of the technology can distinguish a meaningful molecule interaction from: nonspecific aggregation between molecules, interactions due to misfolded proteins, molecules interacting indirectly (e.g., two molecules separately binding to a third molecule), and/or other artifacts. For example, the method can include using a representation (e.g., an embedding) of each molecule and/or pair of molecules as a model input to evaluate experimentally observed hits. Measured abundances (e.g., measured intensities) of an observed interaction and/or other property can be poorly correlated with biological relevance. In an example, the method can include using a representation (e.g., an embedding) of each molecule and/or pair of molecules as a model input to evaluate experimentally observed hits with increased accuracy of hit prioritization.

Second, variants of the technology can reduce biases, such as: molecule abundance bias, measurement bias, technical variation (e.g., batch effects), and/or other biases. For example, the method can include transforming screening plate images using a trained model to ‘clean’ the screening plate images. In another example, the method can use supplemental information (e.g., context for molecules and/or experimental data) to evaluate experimentally observed hits, which can reduce biases.

Third, variants of the technology can be agnostic to the type of experimental data. In examples, multiple types of experimental data can be analyzed, multiple types of molecules (e.g., peptides, small molecules, etc.) can be evaluated, and/or multiple types of molecular interactions (e.g., peptide-small molecule, peptide-peptide, etc.) can be evaluated.

However, further advantages can be provided by the system and method disclosed herein.

### 4. System

The system can include a computing system. The system can optionally include and/or interface with: a database, user interface, assay tools, and/or any other suitable components.

The computing system (e.g., processing system) can include one or more: CPUs, GPUs, TPUs, custom FPGA/ASICS, microprocessors, servers, cloud computing, and/or any other suitable components. The computing system can be local, remote (e.g., cloud-based), distributed, and/or otherwise arranged relative to any other system or module.

The system can optionally include or interface with a database, which can function to store experimental data (e.g., processed and/or unprocessed experimental data), supplemental information, molecules, molecule parameters, representations, scores, training data, and/or any other information. Specific examples of molecule parameters include: molecule sequences (e.g., peptide sequences), molecular structure (e.g., 3D structure of a small molecule and/or peptide; determined using experimental data, computationally determined, etc.), chemical formula, molecular function information, supplemental information, and/or any other information associated with one or more molecules.

The system can optionally include a user interface, which can function to receive one or more inputs (e.g., from a user), display one or more outputs (e.g., model outputs, scores, molecules, etc.), display any other parameters, and/or otherwise function. In a specific example, the user interface can receive experimental data, supplemental information, molecules, molecule parameters, and/or any other information.

The system can optionally include or interface with one or more assay tools (e.g., instruments), which can function to collect (e.g., measure) experimental data. Examples of assay tool types include: mass spectrometers (e.g., configured to perform a mass spectrometry assay), DNA/RNA sequencers, imaging systems (e.g., fluorescence microplate reader), high-throughput screening devices (e.g., including an imaging system such as a fluorescence microplate reader), phage displays, DNA encoded libraries, antibody-based detection systems, cell permeability assays, any quantification-based assay platform, and/or any other assay tool type. Examples of mass spectrometry assays include: affinity purification-mass spectrometry (AP-MS), liquid chromatography-mass spectrometry (LC-MS), time-of-flight mass spectrometry (TOF-MS), gas chromatography-mass spectrometry (GC-MS), capillary electrophoresis-mass spectrometry (CE-MS), ion mobility spectrometry-mass spectrometry (IMS-MS), selected-ion flow-tube mass spectrometry (SIFT-MS), fourier transform mass spectrometry (FT-MS), ion trap mass spectrometry (IT-MS), any other mass spectrometry assay, and/or any other suitable assays. Any mass spectrometry assay can optionally be a tandem mass spectrometry assay (e.g., liquid chromatography-tandem mass spectrometry (LC-MS/MS)). Examples of mass spectrometry (MS) modes that can be used to collect experimental data include: data-dependent acquisition (DDA), data independent acquisition (DIA), tandem mass tag (TMT), and/or any other MS method.

The system can include one or more models, including evaluation models, processing models (e.g., calibration models), molecule encoders (e.g., single molecule encoder, multiple molecule encoder, etc.), other encoders (e.g., experimental data encoder, supplemental information encoder, etc.), image modification models, and/or any other model.

The models can use classical or traditional approaches, machine learning approaches, and/or other approaches. The models can include regression (e.g., linear regression, non-linear regression, logistic regression, etc.), decision tree, latent semantic analysis (LSA), clustering (e.g., k-means clustering, hierarchical clustering, etc.), association rules, dimensionality reduction (e.g., PCA, t-SNE, LDA, etc.), neural networks (e.g., GNN, RNN, CNN, DNN, CAN, LSTM, RNN, FNN, encoders, decoders, deep learning models, transformers, etc.), ensemble methods (e.g., boosting, boostrapped aggregation, stacked generalization, gradient boosting machine method, random forest method, etc.), multimodal models, optimization methods, classification, rules, heuristics, equations (e.g., weighted equations, etc.), selection (e.g., from a library), lookups, regularization methods (e.g., ridge regression), Bayesian methods (e.g., Naiive Bayes, Markov, etc.), instance-based methods (e.g., nearest neighbor), kernel methods, support vectors (e.g., SVM, SVC, etc.), statistical methods (e.g., probability, multiple hypothesis testing, etc.), comparison methods (e.g., matching, distance metrics, thresholds, etc.), deterministics, genetic programs, foundation models (e.g., large language models), hidden Markov models (HMM), and/or any other suitable model. The models can optionally include language-based models and/or vision-based models.

The models can include (e.g., be constructed using) a set of input layers, output layers, and hidden layers (e.g., connected in series, such as in a feed forward network; connected with a feedback loop between the output and the input, such as in a recurrent neural network; etc.; wherein the layer weights and/or connections can be learned through training); a set of connected convolution layers (e.g., in a CNN); a set of self-attention layers; and/or have any other suitable architecture. The models can include less than 10, tens, hundreds, thousands, tens of thousands, hundreds of thousands, and/or any other number of parameters (e.g., weights, biases, etc.). The models can extract data features (e.g., feature values, feature vectors, high-dimensional features, embeddings in a high-dimensional space with hundreds or thousands of dimensions, human-unintelligible features, etc.) from the input data, and determine the output based on the extracted features. However, the models can otherwise determine the output based on the input data.

Models can be trained, learned, fit, predetermined, and/or can be otherwise determined. The models can be trained or learned using: supervised learning, unsupervised learning, self-supervised learning, semi-supervised learning (e.g., positive-unlabeled learning), reinforcement learning, transfer learning, Bayesian optimization, fitting, interpolation and/or approximation (e.g., using gaussian processes), backpropagation, and/or otherwise generated. The models can be learned or trained on: labeled data (e.g., data labeled with the target label), unlabeled data, positive training sets (e.g., a set of data with true positive labels, negative training sets (e.g., a set of data with true negative labels), and/or any other suitable set of data.

Any model can optionally be validated, verified, reinforced, calibrated, or otherwise updated based on newly received, up-to-date measurements; past measurements recorded during the operating session; historic measurements recorded during past operating sessions; or be updated based on any other suitable data.

Any model can optionally be run or updated: once; at a predetermined frequency; every time the method is performed; every time an unanticipated measurement value is received; or at any other suitable frequency. Any model can optionally be run or updated: in response to determination of an actual result differing from an expected result; or at any other suitable frequency. Any model can optionally be run or updated concurrently with one or more other models, serially, at varying frequencies, or at any other suitable time.

However, the system can be otherwise configured.

### 5. Method

As shown in FIG. 1, the method can include: determining a set of molecules S100, determining a representation for the set of molecules S150, and evaluating a property of the set of molecules S400. The method can optionally include: determining experimental data S200, processing the experimental data S250, determining a set of experimental data features S280, determining supplemental information S300, training a model S500, and/or any other suitable steps.

All or portions of the method can be performed by one or more components of the system, using a computing system, using a database (e.g., a system database, a third-party database, etc.), by a user, and/or by any other suitable system. All or portions of the method can be performed automatically, manually, semi-automatically, and/or otherwise performed.

All or portions of the method can be performed in real time (e.g., responsive to a request), iteratively, concurrently, asynchronously, periodically, and/or at any other suitable time. For example, all or portions of the method can be repeated for each molecule in a set, repeated for each molecule interaction, repeated for each observation in experimental data, and/or otherwise iteratively performed.

### 5.1. Determining a Set of Molecules S100.

Determining a set of molecules S100 functions to determine one or more molecules of interest for evaluation. S100 can be performed after S200, before S200, independently from S200 (e.g., without S200), and/or at any other time. Molecules can include a peptide (e.g., a modified peptide, an unmodified peptide, etc.), small molecule, RNA molecule, DNA molecule, protein, proteoform, and/or any other molecule. Molecules can be naturally occurring molecules, modified molecules (e.g., synthetic molecules), and/or any other molecule type.

The set of molecules can include a single molecule or multiple molecules (e.g., a pair of molecules, multiple pairs of molecules, multiple candidate molecules, etc.). In a specific example, the set of molecules can include multiple interacting molecules (e.g., 2, 3, 4, 5, more than 5, etc.). In specific examples, interacting molecules can be protein-protein, protein-small molecule, small molecule-small molecule, and/or any other molecule-molecule interaction. In a first illustrative example, the set of molecules can include a first protein and a second protein. In a second illustrative example, the set of molecules can include a protein and a small molecule.

Multiple sets of molecules can optionally be determined (e.g., wherein S150 and S400 are iteratively performed for each set of molecules). S100 can optionally be iteratively performed (e.g., to determine multiple sets of molecules). For example, S100 can be performed for each molecule of interest in one or more samples, for each observation of interest (e.g., molecule interaction) in the experimental data, for each of a set of candidate molecules, and/or otherwise performed. In a first example, each set of molecules can include a single molecule of interest, wherein multiple molecules of interest (multiple sets of molecules) can be determined. In a second example, each set of molecules can include a pair of molecules, wherein multiple pairs of molecules (multiple sets of molecules) can be determined.

The set of molecules can be manually determined, determined based on user inputs, determined based on the experimental data, determined based on a molecule of interest, determined based on supplemental information, retrieved (e.g., from a database), predetermined, randomly determined, determined using a model, a combination thereof, and/or otherwise determined. In a first example, the set of molecules can include all or a subset of molecules in a sample (e.g., the sample used to acquire the experimental data). In a second example, the set of molecules can be manually specified by a user. In a third example, the set of molecules can be output by an experimental data preprocessing model (e.g., a matching model that matches spectra to molecules). In a fourth example, the set of molecules can be determined based on one or more observations identified in the experimental data (e.g., wherein each observation is mapped to one or more molecules). In a specific example, for a spectrum of interest (e.g., an MS spectrum peak) identified in the experimental data, the set of molecules can include one or more molecules (e.g., a single molecule, a pair of molecules, etc.) associated with that spectrum of interest. In an illustrative example, a set of molecules is determined for each observation (e.g., spectrum of interest) in the experimental data. In a fifth example, the set of molecules can be determined by modifying another molecule (e.g., a random mutation of a molecule, a targeted mutation of a molecule, etc.). In a sixth example, a combination of the previous examples can be used. In a specific example, a molecule of interest can be determined, a set of candidate molecules can be determined (e.g., selected based on the molecule of interest, retrieved from a database, etc.), and the molecule of interest can be iteratively paired with each candidate molecule in the set of candidate molecules to determine multiple pairs of molecules (multiple sets of molecules).

However, the set of molecules can be otherwise determined.

### 5.2. Determining a Representation for the set of Molecules S150.

Determining a representation for the set of molecules S150 functions to encode information associated with the set of molecules. S150 can optionally be iteratively performed for each set of molecules across multiple sets of molecules. The representation can be an embedding (e.g., an image, a vector, etc.) and/or any other representation. Representations for one or more sets of molecules can optionally be precomputed and stored in a database.

The representation for the set of molecules can optionally be determined using one or more molecule encoders (e.g., single molecule encoders, multiple molecule encoders such as a pairwise molecule encoder, etc.). Multiple molecule encoders can optionally be used in parallel and/or in series. Inputs to the molecule encoder can include molecule parameters, including one or more of: one or more molecule sequences (e.g., a peptide sequences), molecular structure (e.g., 3D structure of a small molecule and/or peptide; determined using experimental data, computationally determined, etc.), chemical formula, molecular function information, supplemental information, a representation for one or more molecules, and/or any other information associated with one or more molecules in the set of molecules. Outputs from the molecule encoder can include the molecule representation and/or any other suitable outputs. In specific examples, the molecule encoder can be or include one or more of: GNNs (graph neural networks), RNNs, transformers, language models (e.g., LLMs), geometric CNNs, any autoencoder, and/or any other model. In a specific example, the molecule encoder can include a language model, wherein inputs and/or training data can include evolutionary information (e.g., multiple different sequences across species that have the same shape and/or function). In another specific example, the molecule encoder can include a GNN, wherein inputs and/or training data can include the number of bonds separating atoms of interest in a molecule. In another specific example, the molecule encoder can include a geometric CNN, wherein inputs and/or training data can include the atoms of a molecule that are located within one or more shells (e.g., of predetermined radii) in the molecule's 3D structure. In another specific example, the molecule encoder can include all or a portion of an Evolutionary Scale Model (ESM). In another specific example, the molecule encoder can include all or a portion of an Evo2 model.

In a first variant, for a set of molecules that includes a single molecule of interest, the molecule encoder can include a single molecule encoder that outputs a representation for the (single) molecule of interest.

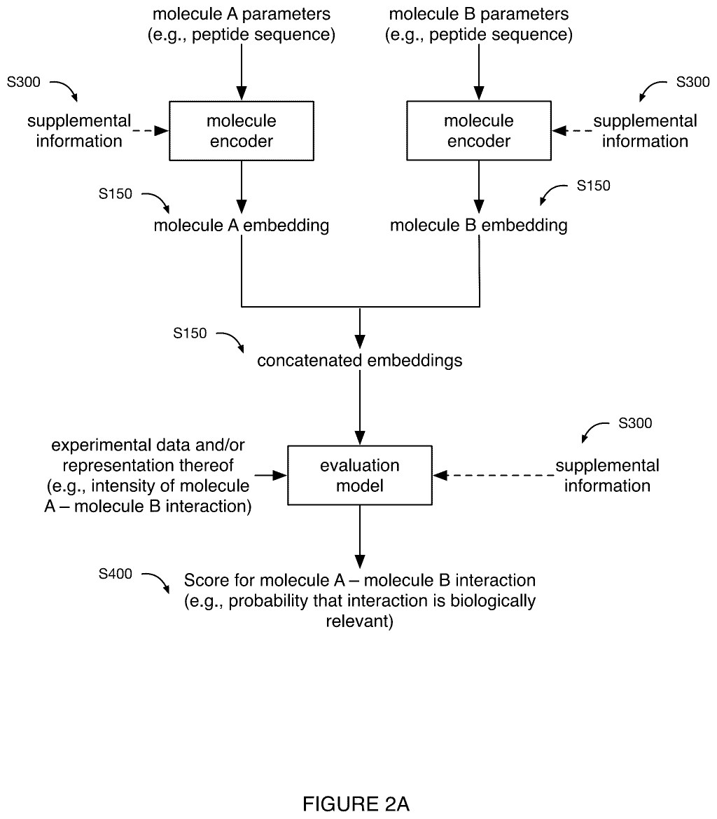

In a second variant, for a set of molecules that include multiple molecules of interest (e.g., a pair of molecules), the molecule encoder can include a multiple molecule encoder (e.g., pairwise encoder) that outputs a representation for the multiple molecules of interest. For example, the molecule encoder can include a pairwise molecule encoder that outputs a representation for a pair of molecules of interest; an example is shown in FIG. 2C. In a first example, the molecule encoder can be a molecule trained to output a single embedding based on one or more molecule parameters (e.g., a sequence) for each molecule in the set of molecules. In a second example, the molecule encoder can use a molecule structure database (e.g., a protein structure database, such as AlphaFold). In a specific example, for a matrix of protein information where the rows are proteins (e.g., protein identifiers) and the columns are amino acids, the molecule encoder can output a pairwise correlation between two proteins. Inputs to the molecule encoder can optionally include co-evolution and/or evolutionary distance between proteins (e.g., as supplementary information). In a third example, the molecule encoder can use a matrix of experiment runs (e.g., a matrix of proteomics experiment runs) to determine correlation (e.g., co-abundance, co-variance, etc.) of two molecules across experiments. In a specific example, for a matrix where the rows are individual experiment runs and the columns are molecules (e.g., proteins), the molecule encoder can output a pairwise correlation between two molecules. Inputs to the molecule encoder can optionally include similarity between experiment runs.

In a third variant, for a set of molecules that include multiple molecules of interest (e.g., a pair of molecules), a single molecule encoder can output an individual representation for each molecule in the set of molecules (e.g., each molecule in the pair of molecules), wherein the individual representations can be used to determine an aggregate representation for the set of molecules. In a first example, the individual representations can be concatenated to generate the aggregate representation; an example is shown in FIG. 2A. In a second example, a multiple molecule encoder (e.g., pairwise molecule encoder) can output the aggregate representation based on the individual representations; an example is shown in FIG. 2B.

However, the representation for the set of molecules can be otherwise determined.

### 5.3. Determining Experimental Data S200.

The method can optionally include determining experimental data S200, which functions to determine data for one or more molecules (e.g., molecules in one or more samples).

The experimental data can include one or more observations associated with one or more sets of molecules (e.g., one or more individual molecules, one or more pairs of molecules, etc.). The observation is preferably a quantified observation, but can additionally or alternatively be a qualitative observation, and/or be otherwise characterized. In a specific example, experimental data can include a (quantified) observation for a property (e.g., activity) of a molecule of interest. In illustrative examples, the property of a molecule of interest can include: a presence and/or abundance of the molecule of interest, a (quantified) interaction between the molecule of interest and a target (e.g., an abundance of interactions between the molecule of interest and the target, a measure of binding affinity, etc.), cell permeability of the molecule of interest, thermostability of the molecule of interest, any experimentally observed hit associated with the molecule of interest and/or any other experimental result for the molecule of interest. In a second specific example, experimental data can include an observation for a property of multiple molecules of interest (e.g., a pair of molecules of interest). In illustrative examples, the property of the multiple molecules of interest can include: a presence and/or abundance of the molecules of interest, a (quantified) interaction between the molecules of interest (e.g., an abundance of interactions between the molecules of interest, a measure of binding affinity, etc.), any experimentally observed hit associated with the molecules of interest, and/or any other experimental result for the molecules of interest.

Experimental data can include interactomics data, proteomics data, metabolomics data, transcriptomics data, genomics data, epigenetics data, any other molecular data, a combination thereof, and/or any other experimental data. Examples of experimental data can include mass spectrometry data (e.g., spectra, spectra intensity, retention times, mass/charge, etc.), other spectra, images (e.g., fluorescence), sequences (e.g., RNA and/or DNA sequences), mass, charge, signals, structure data, and/or any other data. Experimental data can be 1D, 2D, 3D, and/or have any other number of dimensions. Experimental data can be acquired using one or more assay tools and/or assay tool parameters. Experimental data can include one or more data types. Examples of data types can include: spectra, types of spectra (e.g., DDA spectra, DIA spectra, TMT spectra, etc.), sequences, types of sequences (e.g., RNA, DNA, etc.), images, types of images (e.g., fluorescence images), and/or any other data type. Experimental data can be retrieved (e.g., from a database), received (e.g., from a user, from a third-party system, etc.), collected using an assay tool, simulated, predicted, predetermined, manually determined, a combination thereof, and/or otherwise determined.

In a first example, the experimental data can include a spectrum for one or more molecules (e.g., a spectrum for an individual molecule of interest, a spectrum for a pair of interacting molecules, etc.). In a second example, the experimental data can include an abundance of one or more molecules. In a third example, the experimental data includes one or more images. In a specific example, the one or more images can be collected using a high-throughput screening device that includes an imaging system (e.g., fluorescence microplate reader). In a specific example, the one or more images can be image(s) for one or more plates (e.g., the image(s) can correspond to screening plates for high-throughput screening assays). The number of images (e.g., the number of plates) can optionally be between 1-1000 or any range or value therebetween (e.g., 1, 2, 3, 4, 5, at least 2, at least 3, at least 5, at least 10, etc.). Each plate can optionally include a set of wells, where each well on the plate corresponds to (e.g., contains) a set of molecules (e.g., a molecule pair) under evaluation. Multiple wells within a plate and/or across different plates can optionally correspond to different sets of molecules (e.g., for screening multiple molecule pairs). Additionally or alternatively, multiple wells within a plate and/or across different plates can optionally correspond to the same set of molecules (e.g., for repeat evaluation of the same molecule pair). In an example, each image for a plate can include a set of pixels for each well in the set of wells of the plate (e.g., screening plate). The number of pixels corresponding to each well can optionally be between 1-1000 or any range or value therebetween (e.g., 1, 2, 3, 4, 5, etc.). Each pixel can include an intensity value (e.g., fluorescence intensity value). In a specific example, the intensity value of a pixel corresponding to a well can be a measurement of the quantity of the set of molecules (e.g., the molecule pair) contained in that well.

However, experimental data can be otherwise determined.

### 5.4. Processing the Experimental Data S250.

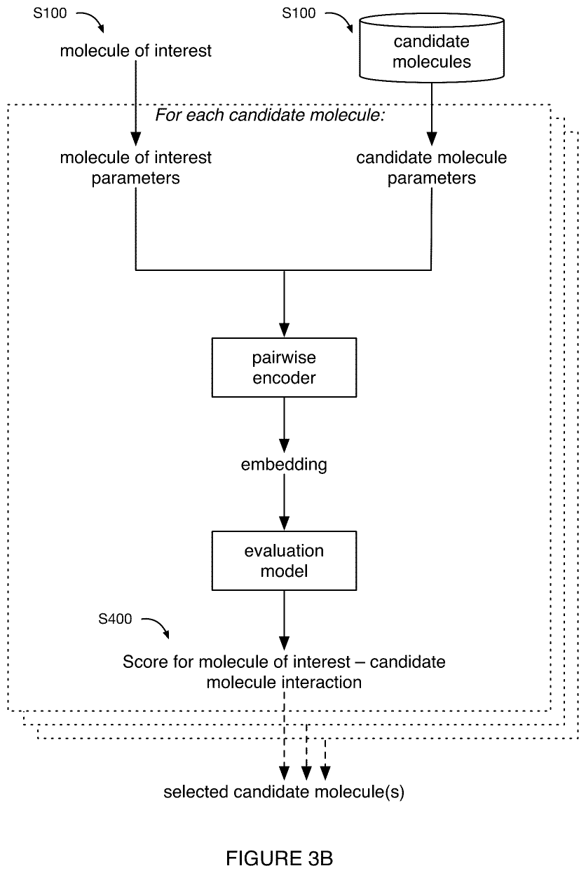

The method can optionally include processing the experimental data S250, which can function to clean the experimental data, reduce biases (e.g., molecule abundance bias, measurement bias, technical variation such as batch effects, etc.), output quantification values, determine confidence levels, determine a representation of the experimental data, and/or otherwise process the experimental data for downstream use. An example is shown in FIG. 6.

Processed experimental data can optionally be or include a representation of the experimental data (e.g., a transformation of the experimental data, an embedding, an image, a matrix, etc.). Examples of processed experimental data include: calibrated experimental data, normalized experimental data, downsampled experimental data, extracted features (e.g., abundances, retention times, etc.), predicted data (e.g., expected values, etc.), synthetic data, and/or any other data. In a first specific example, predicted data can include a predicted map (e.g., an image) of positive and/or negative hits. In a second specific example, predicted data can include an expected value (e.g., expected experimental data value, expected property value, etc.; for each well in a plate). In an illustrative example, expected value(s) can be used for (unprocessed) experimental data value normalization.

In a first embodiment, processed experimental data can include abundance(s) of a set of molecules (e.g., a molecule pair) extracted from the experimental data. In specific examples, abundance can be determined based on spectrum intensity (e.g., peak value, integral, etc.), fluorescence intensity (e.g., peak value, integral, etc.), and/or otherwise extracted from experimental data. In a second embodiment, processed experimental data can include calibrated experimental data. For example, experimental data can be calibrated to reduce noise (e.g., batch and/or plate effects).

Processing the experimental data can optionally include using a processing model (e.g., an experimental data encoder, a calibration model, etc.) to output the processed experimental data. In examples, the processing model can use median polish, B-score, R-score, other statistical methods, neural networks (e.g., CNN), vision-based models, encoders, masked autoencoder-like methods, and/or any other models. In a specific example, the processing model can be or include a trained CNN. In a specific example, the processing model can output processed experimental data. In a specific example, the processing model can ingest experimental data, supplemental information (e.g., well identifier, batch identifier, plate identifier, etc.), molecule parameters, representation(s) of set(s) of molecules, and/or any other model inputs. In variants, using molecule representations can enable the model to be trained to learn different backgrounds for the plate and/or batch effect depending on molecule properties.

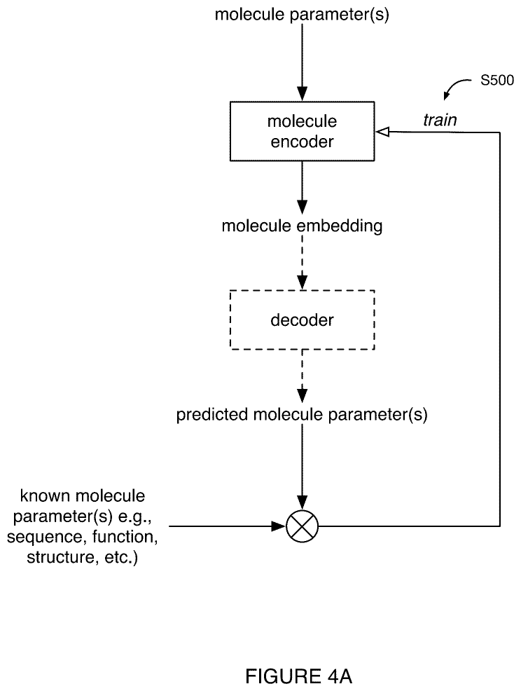

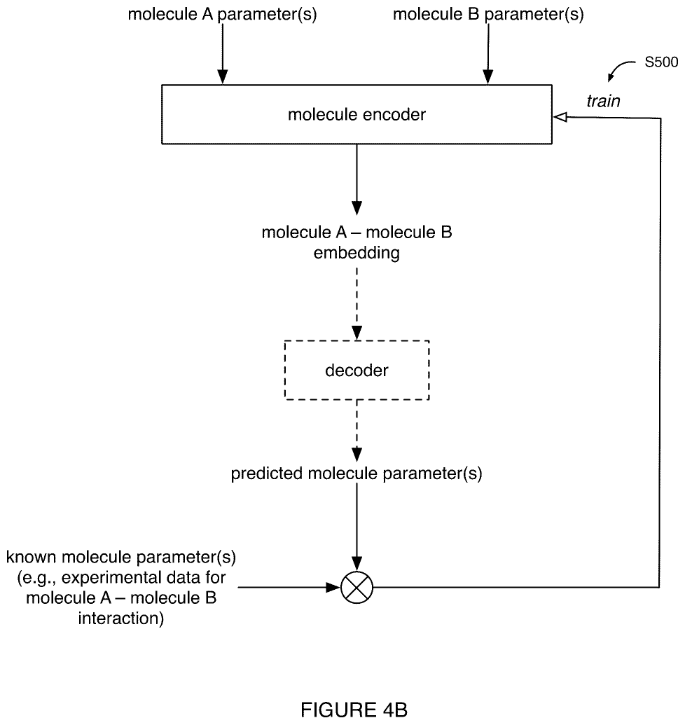

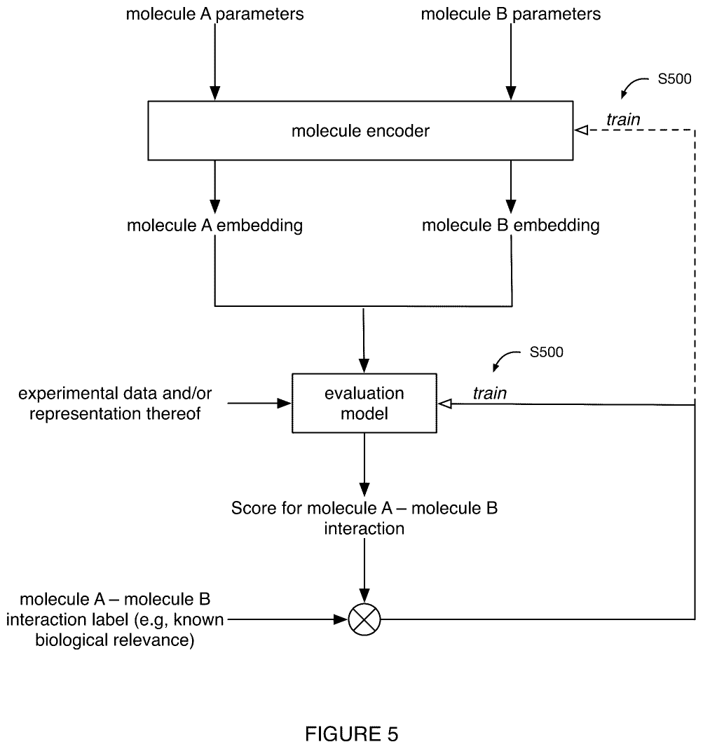

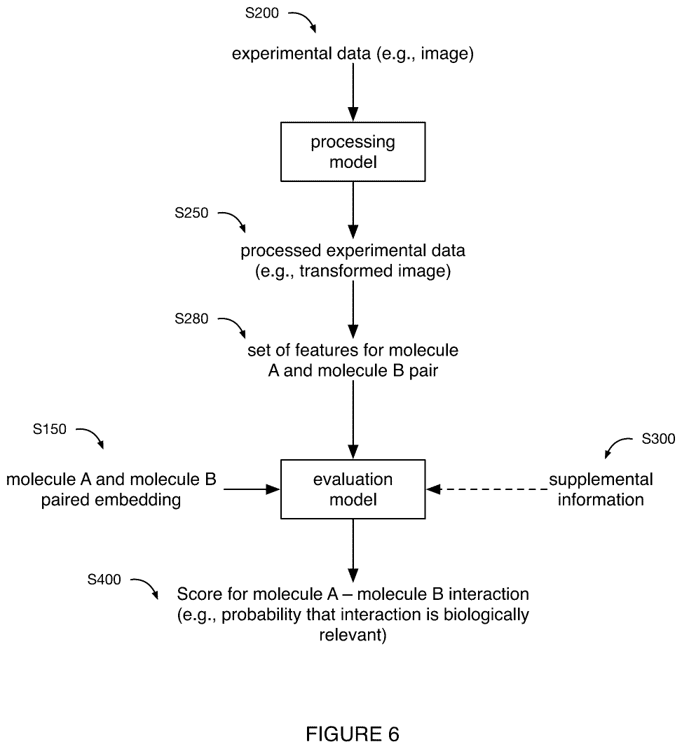

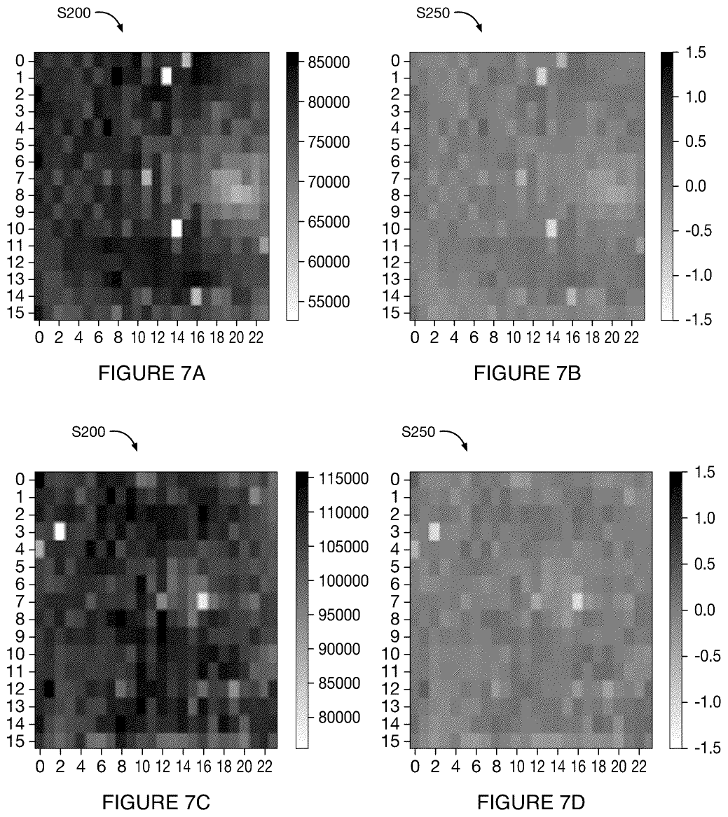

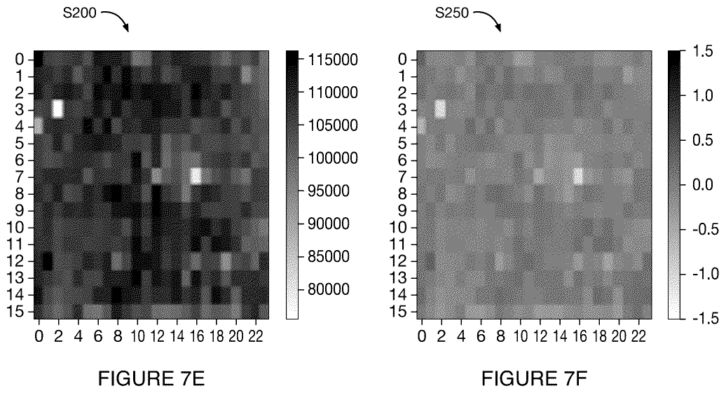

In a first variant, the experimental data includes one or more images, and processing the experimental data includes transforming the one or more images. Examples are shown in FIGS. 7A-7F. For example, when experimental data for a set of molecules (e.g., a set of molecule pairs) includes an image (e.g., an image for a screening plate), a representation of the experimental data can include a transformation of the image (e.g., a transformed image). In an example, inputs to the processing model can include: images for one or more plates (e.g., as described above), supplemental information (e.g., as described below), and/or any other suitable inputs. In an example, outputs from the processing model can include transformed images for the one or more plates, and/or any other suitable outputs. In a specific example, for an image that includes a set of pixels (e.g., a single pixel or multiple pixels) for each well of a plate, the transformed image can include a transformed set of pixels (e.g., a single transformed pixel or multiple transformed pixels) for each well of the plate. In an illustrative example, each pixel corresponding to a well of the plate can be transformed to generate a transformed pixel corresponding to the well of the plate. Each transformed image can optionally be normalized (e.g., where the intensity values within a plate are normalized to a range from 0 to 1).

The processing model can optionally be trained using synthetic training images. For example, a synthetic training image (e.g., each synthetic training image in a set of synthetic training images) can be generated by: generating an image (e.g., a clean image) for a synthetic training plate, and using an image modification model, transforming the image for the synthetic training plate to generate the synthetic training image. The image (e.g., clean image) for the synthetic training plate can optionally function as the label during training of the processing model. For example, the input to the processing model can be the synthetic training image, and the processing model can be trained using a loss function that compares the clean image (e.g., the label) to the transformed image output from the processing model. The image (e.g., clean image) for the synthetic training plate can optionally have randomly distributed ‘hits.’ For example, the image (e.g., clean image) for the synthetic training plate can be generated by randomly distributing high intensity pixels (e.g., corresponding to hits) in the image, wherein each high intensity pixel corresponds to a well in the synthetic training plate. In a specific example, the image modification model can be trained using experimental data (e.g., wherein the image modification model learns patterns from the images in the experimental data). In a specific example, the image modification model can be or include a hidden Markov model. In variants, training the processing model using synthetic training images can enable model training without ground truth experimental data.

In a second variant, the experimental data includes one or more spectra (e.g., mass spectrometry spectra), and processing the experimental data includes determining one or more quantities based on the one or more spectra. In an example, the processed experimental data (e.g., a representation of the experimental data) can include a matrix (e.g., a protein expression matrix), where the rows of the matrix include a first set of molecules (e.g., all human proteins) and the columns of the matrix include a second set of molecules (e.g., bait molecules for a pulldown experiment). In a specific example, the value for each element of the matrix can be the quantity of the molecule pair (the pair corresponding to the row molecule and the column molecule), where the quantity of each molecule pair can be determined based on the spectra. The processed experimental data (e.g., a representation of the experimental data) can optionally include a confidence level for each of the quantities (e.g., for each molecule pair). For example, experimental data for a set of molecule pairs can include one or more spectra acquired using a mass spectrometry device, wherein the processed experimental data (e.g., the representation of the experimental data) can include a quantity and a confidence level for each molecule pair.

In an example, the processed experimental data can be determined using systems and/or methods as described in US Application Ser. No. 19/212,433 filed 19, May 2025, which is incorporated in its entirety by this reference.

Processed experimental data can optionally be used as experimental data and/or be used in addition to unprocessed experimental data in all or parts of the method.

However, experimental data can be otherwise processed.

### 5.5. Determining a set of Experimental Data Features S280.

The method can optionally include determining a set of experimental data features S280, which can function to quantify relevant information in the experimental data for evaluating the property of the set of molecules (e.g., the set of molecules determined in S100). S280 can be determined after S100, after S200, after S250, and/or at any other time. An example is shown in FIG. 6.

The set of experimental data features (e.g., experimental data features and/or values thereof) optionally corresponds to the set of molecules determined via S100. For example, the processed experimental data can include data for multiple sets of molecules (e.g., multiple molecule pairs), wherein the set of experimental data features corresponds to a single set of molecules (e.g., a molecule pair of interest, determined via S100) within the multiple sets of molecules. In an example, a set of experimental data features (e.g., values) can be determined for each molecule pair (e.g., each unique molecule pair) in a set of molecule pairs.

The set of experimental data features can be determined based on experimental data (e.g., determined via S200), processed experimental data (e.g., determined via S250), supplemental information (e.g., determined via S300), the set of molecules (e.g., determined via S100), and/or any other information.

In a first variant, the set of experimental data features for the set of molecules (e.g., a molecule pair of interest) can be determined based on the transformed pixel(s) for each well in one or more plates that correspond to the set of molecules. In a first example, the set of experimental data features can be determined using an image of a single plate. In a second example, the set of experimental data features can be determined using each image of a set of plates, acquired in series (e.g., a first image, a second image, and a third image, acquired in series). In a specific example, the experimental data for a set of molecules (e.g., a set of molecule pairs) can include a first image for a first plate, a second image for a second plate, and a third image for a third plate, wherein the second image is acquired prior to acquiring the image for the screening plate, and wherein the third image is acquired after acquiring the image for the screening plate. In an example, for a plate, the set of experimental data features for a molecule pair of interest can be determined based on the transformed pixel(s) for each well in a subset of the set of wells in the plate (e.g., where the subset of the set of wells are the wells in the plate that contain the molecule pair of interest and/or at least one molecule in the molecule pair of interest). The number of wells in the subset of the set of wells (e.g., the number of wells in the plate that correspond to the molecule pair of interest) can optionally be 1-100 or any range or value therebetween (e.g., at least 2, at least 3, at least 5, etc.). In a specific example, the set of experimental data features for a molecule pair of interest can be determined based on the transformed pixel(s) for each well in a subset of a first set of wells in a first plate (e.g., the wells in the first plate that contain the molecule pair of interest and/or at least one molecule in the molecule pair of interest), the transformed pixel(s) for each well in a subset of a second set of wells in a second plate (e.g., the wells in the second plate that contain the molecule pair of interest and/or at least one molecule in the molecule pair of interest), and the transformed pixel(s) for each well in a subset of a third set of wells in a third plate (e.g., the wells in the third plate that contain the molecule pair of interest and/or at least one molecule in the molecule pair of interest).

In a second variant, the set of experimental data features for a set of molecules (e.g., molecule pair of interest) can be determined based on the value (e.g., quantity) in each element of a quantification matrix that corresponds to the set of molecules. In a specific example, the set of experimental data features for a molecule pair of interest can be determined based on the value(s) for each element in a subset of matrix (e.g., where the subset of the matrix are the elements that correspond to the molecule pair of interest, as determined based on the row and column of the elements). The number of elements in the subset of the matrix can optionally be 1-100 or any range or value therebetween (e.g., at least 2, at least 3, at least 5, etc.).

The set of experimental data features can optionally include statistics. For example, the set of experimental data features can be or include one or more of: mean, median, distribution (e.g., standard deviation), entropy, percentile, confidence level, and/or any other statistical feature. The set of experimental data features can include one or more of: a set of abundance features, a set of reproducibility features, a set of specificity features, and/or any other suitable features.

In a first embodiment, the set of abundance features can include one or more features providing information on the quantity (e.g., intensity) of the set of molecules (e.g., the molecule pair of interest). In a first example, for processed experimental data that includes an image (e.g., transformed image) of intensity values, the set of abundance features can include one or more measures of central tendency (e.g., mean, median, etc.) calculated based on the intensities of the pixel(s) for each well (e.g., across one or more plates) corresponding to the set of molecules. In a second example, for processed experimental data that includes a matrix of quantity values, the set of abundance features can include one or more measures of central tendency (e.g., mean, median, etc.) calculated based on the quantities for each element (e.g., across one or more matrices) corresponding to the set of molecules.

In a second embodiment, the set of reproducibility features can include one or more features providing information on the reproducibility of the quantity of the set of molecules across multiple repeat experimental trials. In a first example, for processed experimental data that includes an image of intensity values, the set of reproducibility features can include one or more measures of variability (e.g., standard deviation, entropy, distribution, etc.) calculated based on the intensities of the pixel(s) for each well (e.g., across one or more plates) corresponding to the set of molecules. In a second example, for processed experimental data that includes a matrix of quantity values, the set of reproducibility features can include one or more measures of variability (e.g., standard deviation, entropy, distribution, etc.) calculated based on the quantities for each element (e.g., across one or more matrices) corresponding to the set of molecules.

In a third embodiment, for a set of molecules (e.g., a molecule pair of interest) that includes a first molecule and a second molecule, the set of specificity features can include: one or more features providing information on the specificity of the quantity of the set of molecules relative to the other sets of molecules that also include the first molecule, and/or one or more features providing information on the specificity of the quantity of the set of molecules relative to the other sets of molecules that also include the second molecule. In a first example, for processed experimental data that includes an image of intensity values, the set of specificity features can include one or more statistics calculated based on the intensities of the pixel(s) for each well (e.g., across one or more plates) corresponding to the first molecule (e.g., corresponding to all sets of molecules that include the first molecule). In a second example, for processed experimental data that includes an image of intensity values, the set of specificity features can include one or more statistics calculated based on the intensities of the pixel(s) for each well (e.g., across one or more plates) corresponding to the second molecule (e.g., corresponding to all sets of molecules that include the second molecule). In a specific example, the set of specificity features can be determined based on the transformed pixel for each well in: a subset wells corresponding to (e.g., containing) the first molecule and the second molecule, a second subset of the set of wells corresponding to (e.g., containing) the first molecule and a third molecule, and a third subset of the set of wells corresponding to (e.g., containing) the second molecule and a fourth molecule. In a third example, for processed experimental data that includes a matrix of quantity values, the set of specificity features can include one or more statistics calculated based on the quantities for each element (e.g., across one or more matrices) corresponding to the first molecule (e.g., corresponding to all sets of molecules that include the first molecule). In a fourth example, for processed experimental data that includes a matrix of quantity values, the set of specificity features can include one or more statistics calculated based on the quantities for each element (e.g., across one or more matrices) corresponding to the second molecule (e.g., corresponding to all sets of molecules that include the second molecule).

The set of experimental data features can optionally be concatenated together (e.g., into a vector of features) for downstream use.

However, the set of experimental data features can be otherwise determined.

### 5.6. Determining Supplemental Information S300.

The method can optionally include determining supplemental information S300, which functions to determine information associated with the experimental data and/or other information associated with the set of molecules. S300 can be performed concurrently with S200, before S200, after S200, and/or at any other time.

Supplemental information can optionally be used as an input into a model (e.g., scoring model, one or more encoder models, etc.). Supplemental information can be retrieved (e.g., from a database such as a public repository), received, collected using an assay tool, simulated, predicted, predetermined, manually determined, a combination thereof, and/or otherwise determined. In a first example, supplemental information can be received as metadata corresponding to the experimental data. In a second example, supplemental information can be determined using a model. In a specific example, a model (e.g., a foundation model) can output contextual information for a molecule. In an illustrative example, the model can output information associated with a network of multiple molecules (e.g., a network of known or high-likelihood interaction candidates for a molecule of interest, a network of similar molecules to a molecule of interest, pairwise representations for the molecule of interest and other molecules, etc.).

Examples of supplemental information can include additional experimental data (e.g., collected using the same or a different assay tool; collected using the same or a different assay tool type and/or mode; data for the same set of molecules or different molecules; etc.), context parameters, predictions (e.g., predicted experimental data, predicted interactions between molecules, etc.), physics information (e.g., physics of molecular fragmentation, molecular structures, molecular interactions, molecular dynamics information, etc.), computational data (e.g., molecular dynamics simulations, computational docking, etc.), a property of interest (e.g., to be evaluated in S400), property type (e.g., individual molecule property, multiple molecule property such as a molecule interaction property, etc.), molecule type (e.g., protein, peptide, small molecule, etc.), evolutionary distance (e.g., between molecules in the set of molecules), and/or other information.

Specific examples of additional experimental data include: measured retention times, other (e.g., adjacent) spectra, precursor time of flight (e.g., ion mobility), precursor m/z, precursor intensity (e.g., peak intensity), peptide sequence length, sample pH and/or other sample environment features, molecular structure data, structural biology experimental data, imaging experimental data, and/or any other experimental data. Specific examples of context parameters can include: assay tool parameters (e.g., type of instrument such as type of mass spectrometry system, make, model, data acquisition mode, instrument settings, etc.), data type, fragmentation parameters (e.g., fragmentation method such as HCD fragmentation, CID fragmentation, etc.), digestion enzyme (e.g., trypsin, non-tryptic digestion, etc.), molecule modifications (e.g., biological modifications, TMT modifications, post-translational modifications (PTMs), any amino acid modification, etc.), molecule charge, molecule length, source protein functional annotation, mutation indicator, sample parameters (e.g., source organism of the sample such as human, plant, microorganism, etc.), batch identifier, plate identifier, well identifier, assay tool identifier, sample location (e.g., well location in a plate), plate sequence (e.g., the order of experimental data collected for each plate), molecule addition sequence (e.g., the order of adding molecules to wells in a plate), similarity between experiment runs, and/or any other context for the experimental data.

However, supplemental information can be otherwise determined.

### 5.7. Evaluating a Property of the set of Molecules S400.

Evaluating a property of the set of molecules S400 functions to evaluate an experimentally observed or predicted property of the set of molecules. In a specific example, S400 can function to evaluate an observation in the experimental data (e.g., an abundance of a pair of molecules interacting, thermostability of a molecule of interest, any other measured property, etc.). In an illustrative example, S400 functions to predict whether an experimentally observed hit is a true hit. In examples, S400 can function to identify high-potential hits (e.g., biologically relevant interactions between molecules), identify candidate molecules with a target property (e.g., candidate molecules with a high-potential for interaction with a molecule of interest), and/or identify other targets. S400 can be performed after S250 and/or at any other time. S400 can optionally be iteratively performed for each set of molecules across multiple sets of molecules.

The property of the set of molecules (e.g., an experimentally observed hit) can optionally be evaluated using an evaluation model. Inputs to the evaluation model can include one or more of: molecule parameters, representation(s) of the set of molecules, processed and/or unprocessed experimental data, one or more sets of experimental data features (e.g., a set of experimental data features corresponding to the set of molecules), supplemental information, representations thereof (e.g., determined using an encoder), and/or any other suitable inputs. In specific examples, inputs to the evaluation model can include: individual representations for each molecule in the set of molecules, an aggregate representation for the set of molecules, a combination thereof, and/or any other inputs. Outputs from the evaluation model can include: a score for the property, a score for all or a portion of the set of molecules (e.g., a score for a candidate molecule in the set of molecules), and/or any other suitable outputs. The score can be qualitative, quantitative, relative, discrete, continuous, a classification, numeric, binary, a ranking (e.g., relative to other scores), and/or be otherwise characterized. In a first illustrative example, the score can represent a likelihood of an observed property being meaningful (e.g., a biologically relevant interaction between molecules in the set of molecules). In a second illustrative example, the score can represent a prediction for a property (e.g., a predicted binding affinity between a molecule of interest and a candidate molecule). In specific examples, the evaluation model can be or include one or more of: regression (e.g., logistic regression), tree-based ensembles, NNs (e.g., a shallow neural network with heavy regularization), cross-attention, contact maps, and/or any other model architecture.

The evaluation model can optionally account for (e.g., correct for) batch effects, plate effects, and/or other experimental noise. In an example, the evaluation model (e.g., CNN, other vision-based models, etc.) can ingest experimental data as an image (e.g., each plate is treated as an image). In a specific example, the evaluation model can determine processed experimental data (e.g., map of positive and negative hits, predicted data, etc.), and output score(s) based on the processed experimental data. In a specific example, the evaluation model can be an ensemble model that includes a processing model (e.g., as a first layer). In an illustrative example, the evaluation model can predict expected values (e.g., expected abundances, expected property values, etc.) for each well in a plate, and can use an observed value/expected value ratio to predict scores (e.g., positive and negative hits).

In an example, the method can include: determining an image for a screening plate, where the image includes a pixel for each well in a set of wells of the screening plate; using a processing model, transforming the image to generate a transformed pixel for each well in the set of wells; and for each molecule pair in a set of molecule pairs: determining a set of experimental data features for the molecule pair based on the transformed pixel for each well in a subset of the set of wells, the subset of the set of wells containing the molecule pair; using a molecule encoder, determining an embedding for molecule pair based on a first sequence corresponding to a first molecule in the molecule pair and a second sequence corresponding to a second molecule in the molecule pair; and using an evaluation model, determining a score for the molecule pair based on the embedding for the molecule pair and the set of experimental data features for the molecule pair. In another example, the method can include: using a processing model, determining a representation of experimental data for a set of molecule pairs (e.g., where the experimental data can be retrieved from a database and/or acquired using an assay tool); and for each molecule pair in the set of molecule pairs: using a molecule encoder, determining an embedding for the molecule pair based on a first sequence corresponding to a first molecule in the molecule pair (e.g., where the first sequence and/or second sequence can be retrieved from a database) and a second sequence corresponding to a second molecule in the molecule pair, and using an evaluation model, determining a score for the molecule pair based on the embedding for the molecule pair and the representation of the experimental data for the set of molecule pairs. In a specific example, the score for the molecule pair can be determined based on the embedding for the molecule pair and a set of experimental data features for the molecule pair, the set of experimental data features for the molecule pair determined based on the representation of the experimental data for the set of molecule pairs. The score for each molecule pair in the set of molecule pairs can optionally be provided to a user (e.g., a user interface can be configured to display the score for each molecule pair).

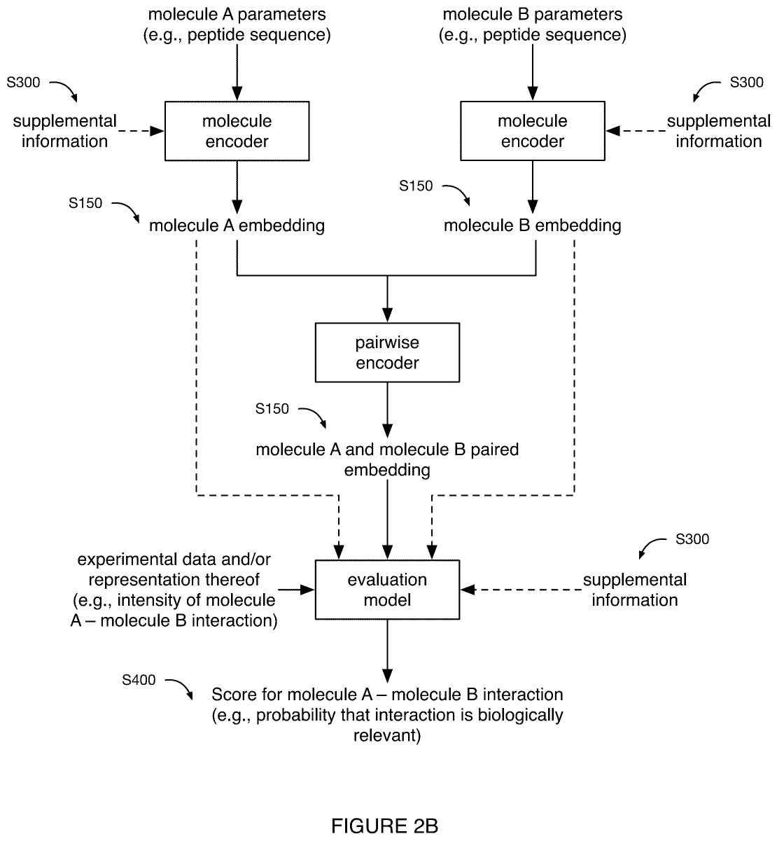

S400 can optionally include ranking multiple properties (e.g., where each property corresponds to a set of molecules) and/or molecules (e.g., ranking individual molecules, ranking sets of molecules, etc.) based on the respective scores. S400 can optionally include identifying (e.g., selecting) a subset of multiple properties and/or a subset of molecules (e.g., the highest scoring: properties, molecules, sets of molecules, etc.) based on the respective scores. In an illustrative example, identified sets of molecules can include pairs of molecules that are predicted to have meaningful interactions. In a specific example, candidate molecules can be ranked and/or identified based on the score for each respective candidate molecule (e.g., the score for each candidate molecule interacting with a molecule of interest). Examples are shown in FIG. 3A and FIG. 3B. In an illustrative example, the identified candidate molecules can include small molecules that are predicted to bind to a molecule of interest.

S400 can optionally include using explainability methods (e.g., transparency, visualization, interpretability, etc.). For example, explainability methods can be used to determine which features of a molecule (e.g., residues) correlate to high binding affinity, which locations of molecules are interacting, and/or other information associated with the score. In a first variant, the evaluation model can include contact maps (e.g., mapping where molecules interact), wherein the contact map can provide explainability. The contact maps can be known, determined based on experimental data, learned, and/or otherwise determined. For example, a contact map can include interaction locations (e.g., in 1D space; locations on the sequence for each molecule in the set of molecules where the molecule is interacting). Contact maps can be used for evaluating all sets of molecules, a portion of multiple sets of molecules (e.g., contact maps can be used when known), or no sets of molecules. In an illustrative example, the evaluation model can output contact map(s) for the set of molecules based on one or more inputs to the evaluation model, and output the score based on: the contact map(s) and, optionally, additional inputs to the evaluation model. In a second variant, attention mechanisms (e.g., cross-attention in the evaluation model) can be used.

However, molecule properties can be otherwise evaluated.

### 5.8. Training a Model S500.

The method can optionally include training a model S500, which functions to train one or more of: evaluation model, molecule encoder, other encoders, and/or any other model. S500 can be performed before S400, after S400 (e.g., using feedback after S400), and/or at any other time. S500 can optionally include fine tuning and/or otherwise retraining models (e.g., finetuning model weights). In an illustrative example, a model can be finetuned for an individual assay tool (e.g., for a specific laboratory) using data acquired with that individual assay tool. S500 can additionally or alternatively include model validation and/or evaluation (e.g., using benchmark and/or performance metrics).

Models can be trained or learned using: supervised learning, unsupervised learning, self-supervised learning, semi-supervised learning, weakly-supervised learning, reinforcement learning, transfer learning, fitting, interpolation, approximation, backpropagation, and/or otherwise trained. Models can be learned or trained on: labeled data (e.g., data labeled with the target label), unlabeled data, positive training sets, negative training sets, and/or any other suitable set of data. Training data can be measured, retrieved from a database (e.g., third-party database; a synthetic library; publicly available database; publications; etc.), augmented (e.g., augmenting positive datasets with negative datasets), simulated, manually determined, randomly determined, and/or otherwise determined. In a specific example, training data can include synthetic negative data (e.g., synthetic molecule sequences derived from biological sequences with a negative label). Training data can optionally include data for multiple species, a single species, multiple different supplemental information values (e.g., experimental data types, property types, molecule types, etc.), the same supplemental information values, a combination thereof, and/or any other data.

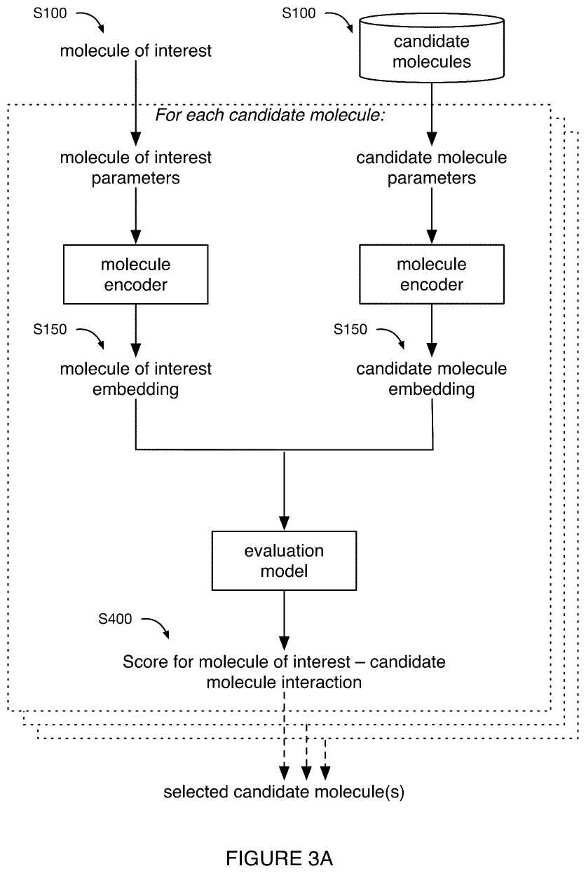

Models can be trained individually, together (e.g., an ensemble model trained using end to end learning), a combination thereof, and/or otherwise trained. For example, encoders (e.g., one or more molecule encoders) and the evaluation model can collectively form an ensemble model, wherein each submodel can be individually trained (e.g., updated) and/or trained together (using end-to-end learning). An example is shown in FIG. 5. In an example, a model can be trained using a loss function (e.g., wherein the loss is backpropagated to the model). In examples, the loss function can be or use: binary cross entropy, a ranking-based loss function (e.g., ‘ground-truth’ data includes ranked hits; the ranking can be determined using: a number of literature citations of the hit, validation assays, and/or any other confidence measure), Siamese models (e.g., contrastive losses), and/or any other loss approach. In a specific example, models can be trained using a small number of labels (e.g., with few shot learning).

Models can optionally be trained using one or more objectives (e.g., using multiple objectives simultaneously). In examples, objectives can include: scores (e.g., rankings), experimental data, representations, molecule parameters, and/or any other targets. In a specific example, the evaluation model can be trained to predict expected values (e.g., as a self-supervised task) and to predict scores (e.g., as a supervised objective).

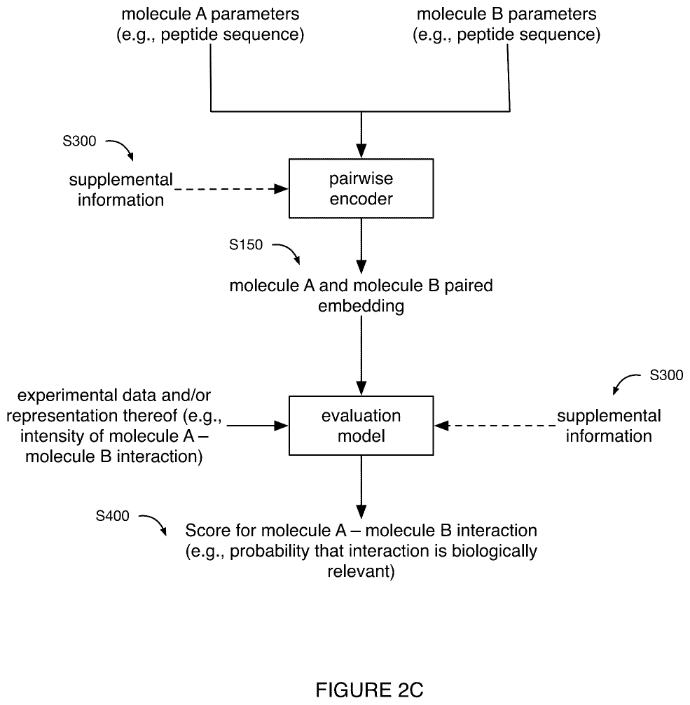

In an example, a molecule encoder (e.g., single molecule encoder and/or multiple molecule encoder) can be partially or fully pretrained; the molecule encoder can then optionally be updated using end-to-end learning. For example, the molecule encoder can be (individually) trained using one or more objectives. In examples, objectives can include molecule parameters, such as: molecule sequence, molecule function, molecule localization, molecule structure, experimental data (e.g., measurements of one or more molecules such as an MS spectrum), physical chemical properties, and/or any other molecule parameter. In a first specific example, the molecule encoder can output a training representation for a single training molecule, a decoder can decode the training representation to predict a molecule parameter for the training molecule (e.g., predicted experimental data, predicted sequence, etc.), and the predicted molecule parameter can be compared to a known molecule parameter (e.g., known experimental data, known sequence, etc.). An example is shown in FIG. 4A. In a second specific example, the molecule encoder can output a training representation for a multiple training molecules (e.g., a pair of training molecules), a decoder can decode the training representation to predict a molecule parameter for the training molecules (e.g., predicted experimental data for an interaction between the training molecules), and the predicted molecule parameter can be compared to a known molecule parameter (e.g., known experimental data). An example is shown in FIG. 4B. In a third specific example, a molecule encoder that includes a language model can be trained using mask token training.

Models can optionally be trained to be specific or general to supplemental information. For example, an individual model can be trained for different values of supplemental information (e.g., an evaluation model can accept different experimental data corresponding to different data types, different property types, etc. ; an ensemble model can include multiple evaluation models pretrained for different data types and/or property types and connected using logic; etc.) and/or multiple models can be trained for different values of supplemental information. Models can optionally be trained for a first supplemental information value (e.g., trained to ingest mass spectrometry data), then retrained for a second supplemental information value (e.g., trained to ingest fluorescence imaging data). In an illustrative example, the evaluation model can be the same for different molecule types (e.g., small molecule and peptide), while the molecule encoder can be retrained for different molecule types. In an illustrative example, the molecule encoder can be the same for different experimental data types and/or property types (e.g., individual molecule property, multiple molecule property, etc.), while the evaluation model can be retrained for different experimental data types and/or property types. Models can optionally be modular. In an illustrative example, a first molecule encoder trained for a first molecule type (e.g., peptides) can be replaced with a second molecule encoder trained for a second data type (e.g., small molecules).

However, models can be otherwise trained.

However, the method can be otherwise performed.

### 5. Specific Examples

A numbered list of specific examples of the technology described herein are provided below. A person of skill in the art will recognize that the scope of the technology is not limited to and/or by these specific examples.

Specific Example 1. A method for evaluating interactions between molecules, the method comprising: determining an image for a screening plate, the image comprising a pixel for each well in a set of wells of the screening plate; using a processing model, transforming the image to generate a transformed pixel for each well in the set of wells; for each molecule pair in a set of molecule pairs: determining a set of experimental data features for the molecule pair based on the transformed pixel for each well in a subset of the set of wells, the subset of the set of wells comprising wells in the screening plate containing the molecule pair, the subset of the set of wells comprising to at least two wells; using a molecule encoder, determining an embedding for molecule pair based on a first sequence corresponding to a first molecule in the molecule pair and a second sequence corresponding to a second molecule in the molecule pair; and using an evaluation model, determining a score for the molecule pair based on the embedding for the molecule pair and the set of experimental data features for the molecule pair; and providing the score for each molecule pair in the set of molecule pairs to a user.

Specific Example 2. The method of Specific Example 1, wherein the set of experimental data features comprise: a set of abundance features, a set of reproducibility features, and a set of specificity features.

Specific Example 3. The method of Specific Example 2, wherein the set of specificity features is determined based on the transformed pixel for each well in: the subset of the set of wells, a second subset of the set of wells, and a third subset of the set of wells, wherein the second subset of the set of wells comprises wells in the screening plate containing the first molecule and a third molecule, wherein the third subset of the set of wells comprises wells in the screening plate containing the second molecule and a fourth molecule.

Specific Example 4. The method of any of Specific Examples 1-3, further comprising: determining a second image for a second screening plate, the second image comprising a pixel for each well in a second set of wells, wherein the second image is acquired prior to acquiring the image for the screening plate; determining a third image for a third screening plate, the third image comprising a pixel for each well in a third set of wells, wherein the third image is acquired after acquiring the image for the screening plate; using the processing model, transforming the second image to generate a transformed pixel for each well in the second set of wells; and using the processing model, transforming the third image to generate a transformed pixel for each well in the third set of wells; wherein the set of experimental data features for the molecule pair is further determined based on: the transformed pixel for each well in a subset of the second set of wells and the transformed pixel for each well in a subset of the third set of wells.

Specific Example 5. The method of any of Specific Examples 1-4, wherein the processing model is trained using a set of synthetic training images, wherein each synthetic training image in the set of synthetic training images is generated by: generating an image for a synthetic training plate by randomly distributing high intensity pixels in the image, each high intensity pixel corresponding to a well in the synthetic training plate; and using an image modification model, transforming the image for the synthetic training plate to generate the synthetic training image.

Specific Example 6. The method of Specific Example 5, wherein the image modification model is trained using experimental data.

Specific Example 7. The method of any of Specific Examples 5-6, wherein the image modification model comprises a hidden Markov model.

Specific Example 8. The method of any of Specific Examples 1-7, wherein the processing model comprises a trained CNN.

Specific Example 9. The method of any of Specific Examples 1-8, wherein the image is collected using a high-throughput screening device comprising an imaging system.

Specific Example 10. The method of any of Specific Examples 1-9, wherein the first molecule comprises a protein, wherein the second molecule comprises at least one of a small molecule or a second protein.

Specific Example 11. A system for evaluating interactions between molecules, the system comprising: a database storing molecule sequences and experimental data for a set of molecule pairs; a processing system configured to: retrieving, from the database, the experimental data for the set of molecule pairs; using a processing model, determining a representation of the experimental data for the set of molecule pairs; for each molecule pair in the set of molecule pairs: retrieving, from the database, a first sequence corresponding to a first molecule in the molecule pair; retrieving, from the database, a second sequence corresponding to a second molecule in the molecule pair; using a molecule encoder, determining an embedding for the molecule pair based on the first sequence and the second sequence; and using an evaluation model, determining a score for the molecule pair based on the embedding for the molecule pair and the representation of the experimental data for the set of molecule pairs; and a user interface configured to display the score for each molecule pair in the set of molecule pairs.

Specific Example 12. The system of Specific Example 11, wherein the experimental data for the set of molecule pairs comprises a spectrum acquired using a mass spectrometry device, wherein the representation of the experimental data for the set of molecule pairs comprises a quantity and a confidence level for each molecule pair.

Specific Example 13. The system of any of Specific Examples 11-12, wherein the experimental data for the set of molecule pairs comprises an image for a screening plate, the image acquired using a high-throughput screening device comprising an imaging system, the image comprising a pixel for each well in a set of wells of the screening plate.

Specific Example 14. The system of Specific Example 13, wherein the representation of the experimental data for the set of molecule pairs comprises a transformation of the image, the transformation of the image comprising a transformed pixel for each well in the set of wells.

Specific Example 15. The system of Specific Example 14, wherein the processing system is further configured to determine a set of experimental data features for the molecule pair based on the transformed pixel for each well in a subset of the set of wells, the subset of the set of wells comprising wells in the screening plate containing the molecule pair, the subset of the set of wells comprising to at least two wells, wherein the score for the molecule pair is determined based on the set of experimental data features for the molecule pair..

Specific Example 16. The system of Specific Example 15, wherein the set of experimental data features comprise: a set of abundance features, a set of reproducibility features, and a set of specificity features.

Specific Example 17. The system of Specific Example 16, wherein the set of specificity features is determined based on the transformed pixel for each well in: the subset of the set of wells, a second subset of the set of wells, and a third subset of the set of wells, wherein the second subset of the set of wells comprises wells in the screening plate containing the first molecule and a third molecule, wherein the third subset of the set of wells comprises wells in the screening plate containing the second molecule and a fourth molecule.

Specific Example 18. The system of any of Specific Examples 13-17, wherein the experimental data for the set of molecule pairs comprises a second image for a second screening plate and a third image for a third screening plate, wherein the second image is acquired prior to acquiring the image for the screening plate, and wherein the third image is acquired after acquiring the image for the screening plate.

Specific Example 19. The system of any of Specific Examples 13-18, wherein the processing model is trained using a set of synthetic training images, wherein each synthetic training image in the set of synthetic training images is generated by: generating an image for a synthetic training plate by randomly distributing high intensity pixels in the image, each high intensity pixel corresponding to a well in the synthetic training plate; and using an image modification model, transforming the image for the synthetic training plate to generate the synthetic training image.

Specific Example 20. The system of Specific Example 19, wherein the image modification model comprises a hidden Markov model.

All references cited herein are incorporated by reference in their entirety, except to the extent that the incorporated material is inconsistent with the express disclosure herein, in which case the language in this disclosure controls.

As used herein, “substantially” or other words of approximation (e.g., “about,” “approximately,” etc.) can be within a predetermined error threshold or tolerance of a metric, component, or other reference (e.g., within +/−0.001%, +/−0.01%, +/−0.1%, +/−1%, +/−2%, +/−5%, +/−10%, +/−15%, +/−20%, +/−30%, any range or value therein, of a reference).

Optional elements, which can be included in some variants but not others, are indicated in broken line in the figures.

Different subsystems and/or modules discussed above can be operated and controlled by the same or different entities. In the latter variants, different subsystems can communicate via: APIs (e.g., using API requests and responses, API keys, etc.), requests, and/or other communication channels. Communications between systems can be encrypted (e.g., using symmetric or asymmetric keys), signed, and/or otherwise authenticated or authorized.

Alternative embodiments implement the above methods and/or processing modules in non-transitory computer-readable media, storing computer-readable instructions that, when executed by a processing system, cause the processing system to perform the method(s) discussed herein. The instructions can be executed by computer-executable components integrated with the computer-readable medium and/or processing system. The computer-readable medium may include any suitable computer readable media such as RAMs, ROMs, flash memory, EEPROMs, optical devices (CD or DVD), hard drives, floppy drives, non-transitory computer readable media, or any suitable device. The computer-executable component can include a computing system and/or processing system (e.g., including one or more collocated or distributed, remote or local processors) connected to the non-transitory computer-readable medium, such as CPUs, GPUs, TPUS, microprocessors, or ASICs, but the instructions can alternatively or additionally be executed by any suitable dedicated hardware device.

Embodiments of the system and/or method can include every combination and permutation of the various system components and the various method processes, wherein one or more instances of the method and/or processes described herein can be performed asynchronously (e.g., sequentially), contemporaneously (e.g., concurrently, in parallel, etc.), or in any other suitable order by and/or using one or more instances of the systems, elements, and/or entities described herein. Components and/or processes of the following system and/or method can be used with, in addition to, in lieu of, or otherwise integrated with all or a portion of the systems and/or methods disclosed in the applications mentioned above, each of which are incorporated in their entirety by this reference.

As a person skilled in the art will recognize from the previous detailed description and from the figures and claims, modifications and changes can be made to the preferred embodiments of the invention without departing from the scope of this invention defined in the following claims.

We claim:

1. A method for evaluating interactions between molecules, the method comprising:
determining an image for a screening plate, the image comprising a pixel for each well in a set of wells of the screening plate;
using a processing model, transforming the image to generate a transformed pixel for each well in the set of wells;
for each molecule pair in a set of molecule pairs:
determining a set of experimental data features for the molecule pair based on the transformed pixel for each well in a subset of the set of wells, the subset of the set of wells comprising wells in the screening plate containing the molecule pair, the subset of the set of wells comprising to at least two wells;
using a molecule encoder, determining an embedding for molecule pair based on a first sequence corresponding to a first molecule in the molecule pair and a second sequence corresponding to a second molecule in the molecule pair; and
using an evaluation model, determining a score for the molecule pair based on the embedding for the molecule pair and the set of experimental data features for the molecule pair; and
providing the score for each molecule pair in the set of molecule pairs to a user.

2. The method of claim 1, wherein the set of experimental data features comprise: a set of abundance features, a set of reproducibility features, and a set of specificity features.

3. The method of claim 2, wherein the set of specificity features is determined based on the transformed pixel for each well in: the subset of the set of wells, a second subset of the set of wells, and a third subset of the set of wells, wherein the second subset of the set of wells comprises wells in the screening plate containing the first molecule and a third molecule, wherein the third subset of the set of wells comprises wells in the screening plate containing the second molecule and a fourth molecule.

4. The method of claim 1, further comprising:
determining a second image for a second screening plate, the second image comprising a pixel for each well in a second set of wells, wherein the second image is acquired prior to acquiring the image for the screening plate;
determining a third image for a third screening plate, the third image comprising a pixel for each well in a third set of wells, wherein the third image is acquired after acquiring the image for the screening plate;
using the processing model, transforming the second image to generate a transformed pixel for each well in the second set of wells; and
using the processing model, transforming the third image to generate a transformed pixel for each well in the third set of wells;
wherein the set of experimental data features for the molecule pair is further determined based on: the transformed pixel for each well in a subset of the second set of wells and the transformed pixel for each well in a subset of the third set of wells.

5. The method of claim 1, wherein the processing model is trained using a set of synthetic training images, wherein each synthetic training image in the set of synthetic training images is generated by:
generating an image for a synthetic training plate by randomly distributing high intensity pixels in the image, each high intensity pixel corresponding to a well in the synthetic training plate; and
using an image modification model, transforming the image for the synthetic training plate to generate the synthetic training image.

6. The method of claim 5, wherein the image modification model is trained using experimental data.

7. The method of claim 5, wherein the image modification model comprises a hidden Markov model.

8. The method of claim 1, wherein the processing model comprises a trained CNN.

9. The method of claim 1, wherein the image is collected using a high-throughput screening device comprising an imaging system.

10. The method of claim 1, wherein the first molecule comprises a protein, wherein the second molecule comprises at least one of a small molecule or a second protein.

11. A system for evaluating interactions between molecules, the system comprising:
a database storing molecule sequences and experimental data for a set of molecule pairs;
a processing system configured to:
retrieving, from the database, the experimental data for the set of molecule pairs;
using a processing model, determining a representation of the experimental data for the set of molecule pairs;
for each molecule pair in the set of molecule pairs:
retrieving, from the database, a first sequence corresponding to a first molecule in the molecule pair;
retrieving, from the database, a second sequence corresponding to a second molecule in the molecule pair;
using a molecule encoder, determining an embedding for the molecule pair based on the first sequence and the second sequence; and
using an evaluation model, determining a score for the molecule pair based on the embedding for the molecule pair and the representation of the experimental data for the set of molecule pairs; and
a user interface configured to display the score for each molecule pair in the set of molecule pairs.

12. The system of claim 11, wherein the experimental data for the set of molecule pairs comprises a spectrum acquired using a mass spectrometry device, wherein the representation of the experimental data for the set of molecule pairs comprises a quantity and a confidence level for each molecule pair.

13. The system of claim 11, wherein the experimental data for the set of molecule pairs comprises an image for a screening plate, the image acquired using a high-throughput screening device comprising an imaging system, the image comprising a pixel for each well in a set of wells of the screening plate.

14. The system of claim 13, wherein the representation of the experimental data for the set of molecule pairs comprises a transformation of the image, the transformation of the image comprising a transformed pixel for each well in the set of wells.

15. The system of claim 14, wherein the processing system is further configured to determine a set of experimental data features for the molecule pair based on the transformed pixel for each well in a subset of the set of wells, the subset of the set of wells comprising wells in the screening plate containing the molecule pair, the subset of the set of wells comprising to at least two wells, wherein the score for the molecule pair is determined based on the set of experimental data features for the molecule pair.

16. The system of claim 15, wherein the set of experimental data features comprise: a set of abundance features, a set of reproducibility features, and a set of specificity features.

17. The system of claim 16, wherein the set of specificity features is determined based on the transformed pixel for each well in: the subset of the set of wells, a second subset of the set of wells, and a third subset of the set of wells, wherein the second subset of the set of wells comprises wells in the screening plate containing the first molecule and a third molecule, wherein the third subset of the set of wells comprises wells in the screening plate containing the second molecule and a fourth molecule.

18. The system of claim 13, wherein the experimental data for the set of molecule pairs comprises a second image for a second screening plate and a third image for a third screening plate, wherein the second image is acquired prior to acquiring the image for the screening plate, and wherein the third image is acquired after acquiring the image for the screening plate.

19. The system of claim 13, wherein the processing model is trained using a set of synthetic training images, wherein each synthetic training image in the set of synthetic training images is generated by:
generating an image for a synthetic training plate by randomly distributing high intensity pixels in the image, each high intensity pixel corresponding to a well in the synthetic training plate; and
using an image modification model, transforming the image for the synthetic training plate to generate the synthetic training image.

20. The system of claim 19, wherein the image modification model comprises a hidden Markov model.
